(19) United States
(12) Patent Application Publication
(10) Pub. No.: US 2025/0281658 A1
(43) Pub. Date: Sep. 11, 2025

(54) Electrically Actuated Portable Scent Dispersion System for Multimedia and Assistive Applications

(71) Applicant: Micheon Eubanks, San Leandro, CA (US)

(72) Inventor: Micheon Eubanks, San Leandro, CA (US)

(21) Appl. No.: 19/218,271

(22) Filed: May 25, 2025

Related U.S. Application Data
(63) Continuation-in-part of application No. 17/687,047, filed on Mar. 4, 2022.

(51) Int. Cl. A61L 9/03

(52) U.S. Cl. CPC A61L 9/03; A61L 9/032; A61L 9/035; A61L 2209/111; A61L 2209/133; A61L 2209/134; A61L 2209/135; A61L 2209/15

(57) ABSTRACT

An electrically actuated portable scent dispersion system configured to emit volatile aromatic compounds in response to electronic control signals. The system comprises a housing containing a scent reservoir and at least one heating element operatively coupled to the reservoir. A control unit receives input signals from an external platform or internal programming and regulates actuation of the heating element to produce a controlled olfactory output. The system is adaptable for integration with multimedia devices, assistive technologies, virtual reality environments, and therapeutic platforms. Embodiments support modular scent cartridges, communication interfaces for synchronization with audiovisual content, and programmable emission profiles. The invention further supports applications in aromatherapy and cognitive therapy, including memory stimulation for patients with neurocognitive disorders.

### BACKGROUND OF THE INVENTION

### Field of Invention

The present invention relates generally to the field of olfactory output systems and electronic scent delivery technologies. More particularly, the invention pertains to a portable, electrically actuated apparatus for the controlled dispersion of scents, designed for integration with multimedia platforms, immersive virtual environments, electronic entertainment systems, and assistive devices for sensory augmentation. The invention further encompasses methods for regulating scent emission in synchronization with digital content or device operations across various consumer, medical, and accessibility-oriented applications.

### BRIEF SUMMARY OF THE INVENTION

The present invention provides a portable, electrically actuated scent dispersion system capable of controlled and programmable release of aromatic compounds. The system comprises a scent-holding medium, one or more electrodes or heating elements, and a control unit configured to initiate and modulate scent emission based on digital input signals. Designed for integration into a wide array of platforms—including virtual reality systems, video games, multimedia content, electronic devices, and assistive technologies—the invention enables enhanced sensory engagement through real-time synchronization of olfactory stimuli with digital experiences.

In addition to its versatile deployment, the invention offers a cost-effective and modular design, allowing for customization of its structural and functional components, such as the scent medium container, labeling materials, and electrode properties. This flexibility enables scalable manufacturing and adaptation for both consumer-grade and specialized industrial or medical applications, facilitating broader access to immersive and accessible scent-based interactions.

### BRIEF DESCRIPTION OF THE FIGURES

FIG. 1 illustrates a scent dispersion activation and delivery process comprising sequential steps including signal receipt (FIG. 1.101), safety validation (FIG. 1.103), cartridge identification (FIG. 1.105), heating element activation (FIG. 1.107), emission regulation (FIG. 1.109), compound dispersion (FIG. 1.111), and system cooldown with data logging (FIG. 1.113).

FIG. 2 illustrates a scent bandage.

FIG. 3 illustrates a diri mount.

FIG. 4 illustrates a diri mailer.

### DETAILED DESCRIPTION

The present invention relates to a portable, electrically actuated scent dispersion system configured for integration with a wide array of electronic platforms and interactive environments. The invention is particularly directed to a modular apparatus for the controlled emission of volatile aromatic compounds through digitally modulated electrical stimulation. The invention is further characterized by its capacity for programmable operation, minimal power consumption, and adaptability to various host environments, including but not limited to multimedia systems, immersive virtual or augmented reality devices, medical instrumentation, assistive technologies for sensory-impaired users, and general-purpose electronic interfaces.

At its core, the invention comprises a discrete hardware unit including a scent containment assembly, one or more thermally responsive actuating elements (e.g., electrodes or resistive heating components), and a control module equipped to receive and interpret electronic signals for regulating emission events. The system is operatively configured to receive either hardwired or wireless input instructions, such that the timing, duration, and intensity of scent dispersion may be synchronized with associated media or user interactions. The invention may optionally comprise onboard memory or firmware capable of storing emission profiles, sensory sequences, or threshold-based activation triggers, thereby enabling context-aware or event-driven olfactory augmentation.

The apparatus may be embodied in a variety of physical forms, including standalone plug-in devices, wearable accessories, embedded modules within electronic hardware, or attachable peripherals capable of integration into existing consumer or medical devices. The invention permits the interchangeability of scent mediums, including solid-phase, gel-based, or encapsulated liquid carriers, housed within thermally stable, removable cartridges or reservoirs. Additionally, the system permits multi-scent functionality, allowing for sequential or simultaneous activation of multiple aroma profiles through isolated actuation pathways or spatially distributed heating zones.

Furthermore, the system is constructed to provide a user-safe interface, minimizing thermal exposure to exterior surfaces, employing insulating housings and fail-safe circuitry to prevent accidental overactivation or leakage. The control logic may further comprise feedback mechanisms capable of regulating current or temperature in response to environmental variables or internal diagnostics. The invention may also include indicators, such as LEDs or haptic feedback modules, to provide visual or tactile cues corresponding to operational states.

The scope of the invention encompasses both the physical hardware and the associated methods of controlling, coordinating, and deploying the scent dispersion events across a plurality of devices and operating contexts. In preferred embodiments, the system leverages existing communication protocols (e.g., Bluetooth, USB, Wi-Fi, IR, or proprietary signaling schemes) to enable seamless interaction with host platforms. In other embodiments, the system may include integrated software libraries or middleware to facilitate API-level integration with multimedia applications, digital content delivery frameworks, or assistive operating systems.

The technical advantages of the invention include, without limitation, enhanced immersion through multisensory interaction, low-cost deployment in consumer and clinical settings, modular scalability, and the capacity to augment traditional audiovisual content with dynamic olfactory experiences. Additionally, the invention enables small-scale producers or content developers to design and distribute scent-enabled media without reliance on proprietary or high-cost infrastructure. The invention thereby addresses long-standing deficiencies in the field of digital olfaction, which has historically been limited by bulky hardware, insufficient programmability, and narrow platform compatibility.

The scent dispersion system described herein is comprised of a modular assembly of physical and electronic components configured to cooperate in facilitating controlled olfactory output in a variety of operating environments. The system's physical architecture is defined by a housing unit that may be manufactured in a range of geometries suitable for application-specific integration, including but not limited to rectangular, cylindrical, or planar forms. The housing may be constructed from thermally resistant, non-conductive materials such as acrylonitrile butadiene styrene (ABS), polycarbonate, ceramic composites, or medical-grade silicone to ensure both electrical insulation and user safety during device operation.

The housing contains a scent reservoir assembly adapted to accommodate a volatile aromatic medium in any of several physical states, including solid-phase wafers, gel-based matrices, impregnated fibrous substrates, or encapsulated liquid solutions. In preferred embodiments, the scent reservoir is removably coupled to the housing to allow for replacement, replenishment, or reconfiguration by the end user. The reservoir may be sealed within an internal cartridge or module, fabricated with a material capable of withstanding localized heating without structural degradation or chemical reaction with the stored aromatic compound.

A primary feature of the device architecture is the actuation mechanism for scent emission, which comprises one or more thermally responsive elements. These elements may include resistive heating coils, printed conductive traces, or metal oxide electrodes embedded in or adjacent to the scent reservoir. When energized, the actuating element elevates the temperature of the aromatic medium past its volatilization threshold, causing a controlled release of scent molecules into the surrounding environment. In some embodiments, thermal conduction is achieved via direct contact between the heating element and the scent medium, while in others, a thermally conductive intermediary (e.g., a metallic or ceramic transfer plate) is employed to ensure uniform heating.

The electrical subassembly includes a printed circuit board (PCB) hosting the microcontroller or digital logic circuitry responsible for device management. The microcontroller may be configured with embedded firmware to process external input signals, execute pre-programmed scent release profiles, regulate power delivery to heating elements, and monitor internal conditions such as temperature, current draw, or cartridge status. The PCB is electrically coupled to a power source, which may be a replaceable battery, a rechargeable lithium-ion cell, or a tethered connection to an external power supply via USB-C, micro-USB, or other standardized interface.

The scent dispersion system may include one or more communication modules adapted to receive external control signals. These may include, without limitation, Bluetooth Low Energy (BLE) transceivers, Wi-Fi adapters, infrared (IR) receivers, near-field communication (NFC) chips, or hardwired serial communication ports (e.g., UART, I2C, SPI). The system may also include sensor modules, such as ambient temperature sensors, motion detectors, humidity sensors, or proximity sensors, to provide contextual inputs that influence scent emission behavior.

Mechanical features of the device may include mounting brackets, adhesive surfaces, magnetic backings, or wearable attachments such as straps or clips. These features enable the apparatus to be affixed to or embedded within a broad range of host devices, including but not limited to head-mounted displays, mobile phones, desktop enclosures, automotive dashboards, or clinical instrumentation. The structural design may further comprise airflow guidance channels, vented apertures, or micro-fan assemblies configured to direct the volatilized aromatic compounds toward the user or target region with minimal dissipation.

In embodiments requiring multi-scent functionality, the housing may include multiple reservoir compartments, each coupled to a dedicated or selectively addressable actuation unit. This configuration allows for either sequential or concurrent activation of different scents, which may be algorithmically combined to produce composite olfactory experiences. Switching between scent modules may be achieved through electronically actuated valves, multiplexed control circuitry, or movable mechanical components such as sliding trays or rotating carousels.

The labeling or identification system for scent cartridges may include visual or electronic means to facilitate user interaction. For instance, cartridges may feature printed QR codes, RFID tags, or EEPROM identifiers to allow the control unit to recognize the specific scent, check expiration dates, or enforce compatibility restrictions. In some embodiments, the system may alert the user via visual indicator, sound cue, or host platform notification when a cartridge requires replacement or is improperly seated.

Additionally, the structural design of the invention includes thermal insulation features to minimize heat transfer to the device exterior and maintain safe surface temperatures during extended operation. This may include layered insulation within the housing walls, use of phase-change materials to absorb thermal energy, or implementation of thermal cutoff switches in proximity to the heating element. The invention may further incorporate safety enclosures, sealing gaskets, or tamper-resistant housings to comply with regulatory and industry standards for consumer electronics or medical devices.

The structural configuration of the invention is engineered to provide robust, safe, and scalable scent emission capability through the coordinated integration of modular hardware components. The architecture enables flexible adaptation across multiple use cases while preserving core functionalities related to scent containment, thermal actuation, signal processing, and physical integration with external systems.

The functional operation of the electrically actuated portable scent dispersion system is predicated upon the controlled initiation, modulation, and termination of scent emission events through digital signal processing and thermal actuation. Upon receipt of a qualified activation signal—whether generated internally by a pre-programmed routine or externally via communication interface—the control unit initiates a sequence of operations designed to safely and precisely volatilize the selected aromatic compound.

The control unit, implemented by means of a microcontroller, digital signal processor (DSP), or equivalent embedded logic system, is programmed with a firmware architecture capable of interpreting signal inputs and generating command outputs according to predetermined logic rules or adaptive algorithms. The incoming signals may originate from a host device, a user interface, a remote application, or an autonomous sensor, and may be encoded to specify various operational parameters including, but not limited to, duration of actuation, target temperature, scent selection (in multi-reservoir configurations), emission pattern, and synchronization cues.

Upon validation of the input command, the control unit initiates a timed power delivery cycle to the designated heating element. The power delivery is regulated by means of pulse-width modulation (PWM), current-limiting circuitry, or other closed-loop control methods to achieve and maintain a specific actuation temperature. Temperature monitoring may be conducted via onboard thermistors, resistance temperature detectors (RTDs), or integrated temperature sensors within the heating assembly. The control logic ensures that the heating element operates within safe thermal boundaries, and may automatically terminate or throttle power delivery upon detection of temperature anomalies or excessive duty cycles.

In preferred embodiments, the heating profile is optimized to correspond with the physicochemical properties of the aromatic medium, such that volatilization occurs without combustion, degradation, or excessive thermal inertia. The scent medium is thereby transformed from a latent state into an active olfactory emission through thermal excitation, with the released aromatic molecules dispersed into the surrounding air by natural convection, active airflow, or proximity-based diffusion. In configurations employing a micro-fan or air-movement component, the fan speed may be dynamically adjusted in parallel with the heating element to control scent distribution radius and concentration gradient.

For applications involving multimedia synchronization, the scent emission event may be aligned with time-coded markers or trigger signals embedded in video, game, or virtual environment content. The device may interface with the host platform through standardized APIs or middleware that communicate scent event metadata, enabling real-time alignment of olfactory output with sensory stimuli such as visuals, audio, or haptic effects. In such cases, the system may function as a peripheral rendering unit responsive to runtime content cues, thereby enhancing user immersion.

In multi-scent configurations, the control unit may store or retrieve a scent mapping profile correlating input commands with specific reservoirs or emission channels. Upon activation, the appropriate heating element is energized, and optionally, the other channels are placed in a standby or lockout mode to prevent unintended cross-contamination. In certain embodiments, scent blending may be implemented through simultaneous actuation of multiple heating elements, each associated with a distinct aromatic compound, to create complex olfactory scenes.

The device may also support operational modes wherein scent emission is triggered by user behavior or environmental conditions. For instance, proximity detection (via IR or ultrasonic sensors), gesture recognition (via optical sensors), or biometric input (e.g., heart rate or galvanic skin response) may serve as trigger mechanisms. The firmware may include conditional logic to evaluate input states and determine if emission conditions are satisfied. For example, scent release may be suppressed in high-temperature environments, during battery conservation mode, or when motion is not detected for a predefined interval.

Additionally, the control logic may accommodate learning algorithms or usage pattern recognition to optimize scent delivery. Over time, the device may store emission history data and adjust operational parameters to reduce redundancy, conserve power, or align with user preferences. Firmware updates may be applied over-the-air (OTA) or via a wired interface to update scent profiles, expand feature sets, or correct performance anomalies.

The operational cycle concludes with the deactivation of the heating element, followed by a cooldown phase managed through natural dissipation or active cooling. The control system may enforce a minimum cooldown interval to prevent consecutive overheating and to protect the longevity of both the scent medium and heating apparatus. Diagnostic routines may be executed post-cycle to assess operational integrity, record system metrics, and alert the user to maintenance needs or cartridge status.

The scent dispersion system achieves precise, responsive, and context-aware operation through a sophisticated integration of electronic control, thermal regulation, and communication interfacing. The functional design ensures repeatable and safe scent activation while maintaining flexibility for integration into diverse technological ecosystems and usage contexts.

The scent dispersion system disclosed herein is engineered for compatibility with a broad spectrum of external platforms and host devices, thereby enabling synchronized or responsive olfactory output across multiple interactive domains. Integration mechanisms are implemented through a combination of hardware-level interfaces, communication protocols, software APIs, and logical control structures that allow the system to interoperate with third-party electronics, computing systems, entertainment platforms, and assistive or medical technologies.

At the hardware interface level, the system may include one or more communication ports or wireless transceivers to enable data exchange and control signaling between the scent device and the host platform. In preferred embodiments, standardized interfaces such as USB-C, USB 2.0/3.0, micro-USB, or auxiliary 3.5 mm jack connectors may be used for tethered communication and power supply. In alternative embodiments, wireless protocols such as Bluetooth Low Energy (BLE), Wi-Fi (802.11 family), Zigbee, Near Field Communication (NFC), or proprietary RF transmission schemes may be employed to enable remote control, mobile integration, or mesh-networked operation.

The invention further comprises firmware and/or embedded software libraries configured to interpret external control signals originating from host devices. These signals may be provided in a variety of formats, including digital logic pulses, serialized command strings, API calls, or event-based triggers embedded within media playback systems. To facilitate seamless integration, the scent dispersion system may expose a set of programmable interfaces-either through direct software development kits (SDKs) or middleware translation layers-enabling developers to incorporate olfactory control into their applications without requiring hardware-specific coding.

In multimedia environments, such as virtual reality (VR), augmented reality (AR), video games, and cinematic playback, the scent dispersion system may operate as a peripheral output device synchronized with audiovisual content. The system may interface with game engines (e.g., Unity, Unreal Engine), VR platforms (e.g., Oculus SDK, OpenXR), or content delivery systems through real-time data exchange. Scent events may be indexed within a content timeline and triggered by specific cues (e.g., frame markers, user interaction points, spatial orientation, or environmental changes within the digital environment). The invention may also support adaptive response modes, wherein scent intensity or duration is modulated based on gameplay variables, environmental complexity, or user engagement metrics.

Integration with assistive technologies may be implemented to support accessibility use cases, such as sensory augmentation for visually impaired users. For example, the invention may be paired with digital reading systems or haptic feedback devices to provide scent-based cues corresponding to textual elements, scene changes, or emotional tones in audiobooks, Braille-based devices, or screen readers. In such implementations, the scent system functions as an auxiliary sensory output, enhancing comprehension, orientation, or emotional resonance for users with sensory limitations.

In medical or therapeutic settings, the scent dispersion system may be integrated with diagnostic equipment, biometric monitoring platforms, or therapeutic protocols. For instance, in Alzheimer's or dementia treatment applications, the device may be paired with memory stimulation systems, wearable biometric sensors, or behavioral assessment software to deliver targeted scents intended to trigger memory recall or emotional responses. Integration may also include data feedback loops, wherein the user's physiological responses (e.g., heart rate variability, galvanic skin response, or EEG patterns) are used to dynamically adjust scent profiles or delivery intervals.

The system may further integrate with mobile applications operating on iOS, Android, or other mobile operating systems. A companion application may provide a graphical user interface (GUI) for selecting scent profiles, adjusting emission settings, scheduling events, or enabling geolocation-based triggers. The application may also allow the user to access usage logs, receive firmware updates, or participate in content experiences augmented with scent. Cloud-based integration may enable remote control, content delivery, or user preference synchronization across multiple devices.

Additionally, the scent system may support voice assistant integration (e.g., Amazon Alexa, Google Assistant, Apple Siri) by exposing functions via voice-enabled APIs or smart home protocols. This allows users to initiate or control scent emission through natural language commands, enhancing accessibility and ease of use. Integration with smart home ecosystems (e.g., Apple Homekit, Samsung SmartThings, or Google Home) may further permit automated routines, such as releasing specific scents during certain times of day, environmental conditions, or alongside lighting and sound effects.

From a systems engineering perspective, the invention may function either as a master controller or slave device within a broader hardware topology. In master mode, the scent device may dictate timing and emission parameters based on internal logic or sensor feedback. In slave mode, the device responds exclusively to control signals from an external host. Communication handshaking, error detection, and status reporting protocols may be implemented to ensure reliable bidirectional interaction. Feedback mechanisms may include acknowledgments, status flags, or diagnostic data transmitted back to the host device to confirm successful actuation or signal error states (e.g., cartridge empty, temperature fault, communication timeout).

To facilitate cross-platform compatibility, the invention may employ middleware translators or device abstraction layers that map generalized commands from diverse host systems into device-specific control logic. For instance, a media player running on a smart TV may issue a generic “activate scent profile A” command, which is translated by the scent device's onboard interpreter into a sequence of operations involving power regulation, heating element selection, and fan actuation. Such abstraction enables a single hardware platform to support a broad array of integrations without requiring modification to core firmware.

In all integrations, the device maintains internal safety protocols and access controls to prevent unauthorized or unsafe operation. Authentication mechanisms may be implemented for API access, firmware updates, or mobile app pairing. These may include token-based authentication, encrypted command structures, or device whitelisting to prevent misuse or unintended actuation in sensitive environments.

The scent dispersion system is engineered for comprehensive interoperability with a diverse range of electronic platforms, enabling scalable, secure, and responsive olfactory augmentation. Its modular architecture, communication flexibility, and programmable logic control facilitate robust integration into both consumer-facing and clinical-grade technological ecosystems, thereby maximizing the functional utility and commercial applicability of the invention.

The invention as described in the foregoing sections encompasses numerous alternative embodiments and configurations, each capable of achieving the stated objectives with varying degrees of complexity, specialization, and platform integration. These alternative embodiments fall within the scope of the disclosed invention and are considered part of its protected subject matter, regardless of variations in component arrangement, control architecture, or end-use application.

In one class of alternative embodiments, the scent reservoir structure may be modified to accommodate different physical forms of the aromatic compound, including microencapsulated liquid oils, dry powder dispersions, phase-change waxes, or volatile gels. The housing may be adapted to provide differential thermal insulation for high-temperature materials, or to include microfluidic conduits for liquid transport to a heating zone. In some embodiments, the scent media may be held in interchangeable cartridges or ampoules sealed to prevent degradation and equipped with unique identifiers such as RFID tags or barcodes for automatic recognition by the device.

The heating mechanism may likewise be varied. While preferred embodiments utilize resistive heating elements, alternative configurations may employ inductive heating, microelectromechanical system (MEMS)-based thermal actuators, piezoelectric elements, or chemical heating packs activated electronically. The activation method may also include energy-efficient pulse heating or staged pre-heating to allow for rapid scent emission upon trigger without continuous thermal loading.

The control system may be implemented using different levels of computational complexity, ranging from basic microcontroller-based logic to more advanced microprocessors running embedded Linux or real-time operating systems (RTOS). In embodiments supporting artificial intelligence or machine learning, the control logic may include predictive emission algorithms based on user behavior, biometric feedback, or environmental conditions, allowing dynamic customization of scent patterns. These systems may also include learning-based suppression to reduce scent fatigue through habituation avoidance logic.

Physical form factors may vary widely depending on deployment context. For instance, the device may be miniaturized for integration into a smartphone case, a wearable wristband, or an eyeglass attachment. In other embodiments, the system may be embedded into furniture, automobiles, healthcare devices, or environmental control units such as HVAC systems. Mounting mechanisms may include adhesive pads, magnetic couplings, clip-on brackets, or internal modular interfaces compatible with consumer electronic products.

In addition to entertainment and assistive applications, the invention is adaptable for therapeutic and clinical uses. In aromatherapy-focused embodiments, the device may be preloaded with essential oils or therapeutic fragrance profiles selected to support stress reduction, sleep regulation, mood enhancement, or physiological relaxation. Scent profiles may be user-selectable via a mobile interface, or scheduled in coordination with circadian cycles, guided meditation routines, or biometric data. The device may further include dosage control to ensure safe and consistent therapeutic exposure.

In neurological treatment applications, the invention may be employed as part of memory rehabilitation or cognitive stimulation therapies, particularly for patients diagnosed with Alzheimer's disease, dementia, or other neurocognitive disorders. Clinical studies have demonstrated the potential of olfactory stimuli to elicit memory retrieval and emotional responses in patients with impaired recall. In these embodiments, the scent dispersion system may be paired with audiovisual or tactile cues, and programmed to release specific scents known to correlate with individual memory anchors or emotional states. The control system may be integrated with a care management platform that logs patient responses and adjusts therapy parameters accordingly.

To support such medical use cases, the device may be constructed in compliance with regulatory standards for bio-compatibility, sterility, and electromagnetic compatibility (EMC). Housing materials may be FDA-grade or ISO-certified for clinical environments. Emission cycles may be validated through software safety protocols, and the system may include redundant thermal shutdowns, error logging, and audit-capable operation tracking. In certain embodiments, data generated during therapy sessions may be transmitted to a cloud-based platform for review by medical professionals, with patient privacy protected via secure communication standards and data encryption.

Other variations within the scope of the invention include user personalization features, such as adaptive scent sequencing based on user profiles, region-specific scent libraries, and culturally contextual olfactory content. The system may include over-the-air provisioning of new scent profiles, firmware updates for expanding functional capabilities, or blockchain-based scent media licensing frameworks for secure content distribution.

Furthermore, the device may operate in environments where scent output must be selectively restricted or modulated due to external regulations, such as aircraft cabins, hospitals, or workplaces. In such cases, geofencing or device-to-device communication may enable context-based suppression or alteration of emission parameters. These embodiments may also include alerting systems to inform users or administrators of restricted operation zones, with override capabilities subject to administrator credentials.

The invention admits a wide range of alternative embodiments in structure, function, and application, all of which retain the core inventive concept of an electrically actuated, portable, and programmable scent dispersion system. These embodiments demonstrate the invention's adaptability for consumer, industrial, therapeutic, and medical sectors, while maintaining a unified design philosophy grounded in safety, modularity, and integrative versatility. All such variations, including combinations of the foregoing, are deemed to be within the scope of the present invention as claimed.

In further embodiments, the invention may be implemented in a wearable configuration in which the scent modules—referred to as “Diris”—are arranged along a flexible substrate configured as an adhesive bandage or skin-mounted patch. In such embodiments, the Diris are operatively coupled to a network of positive and negative electrical conductors embedded within or printed onto a flexible circuit board or conductive film layer. The bandage structure may adhere to the skin using medical-grade adhesive and may include a remote or adjacent power supply, such as a rechargeable battery, USB connection, or solar cell. The electrical input may be routed through conductive wires, gold fingers, or other connection means, with current regulation provided by an integrated microcontroller. Each Diri may be individually addressable, and may be coated or enclosed in heat-resistant material such as graphene or silicone to ensure safe operation when in contact with skin.

Additional embodiments contemplate packaging and transport systems for the Diris, including a scent cartridge mailer constructed of cardboard, paper, foam, or other suitable protective materials. The mailer includes internal dividers or compartments to maintain physical separation between cartridges and may be manufactured with sufficient flexibility to allow passage through automated postal sorting systems. The invention further discloses mounting mechanisms whereby Diris are fixed onto electrically active posts within cartridges, wherein the posts function as positive and negative terminals for actuating the scent modules. A label ribbon, optionally affixed to one or both posts, serves both to collect molten or emitted material from the Diri during operation and to display scent-identifying information. This ribbon may be composed of any material compatible with both labeling and scent residue collection, and may form part of a modular or swappable cartridge assembly.

Furthermore, the scope of scent source materials is extended to include layered waxes, photo paper, printable paper media, and inkjet-applied wax compositions. In some configurations, scented wax is deposited via inkjet techniques onto substrates for later activation using electrical heating elements, forming part of a multi-layered emission surface. These emission media may be integrated into static displays, print materials, or media-enhanced books. Particular attention is given to the use of scent cartridges in accessibility-focused applications, including tactile or audio books for the blind, wherein specific scent profiles may be embedded at predefined intervals or content points. The core substrate for Diris may include absorbent or porous media such as aerogel or expanded styrofoam, saturated with essential oils, fragrance solutions, or other liquid-phase aromatic carriers.

All of the foregoing embodiments, whether structural, functional, or material in nature, are considered within the scope of the present invention. These enhancements serve to broaden the applicability of the invention across wearable technology, distribution logistics, print media integration, and sensory accessibility solutions, and are contemplated to be interoperable with the previously described scent control, activation, and integration systems.

### DETAILED DESCRIPTION OF FIGURES

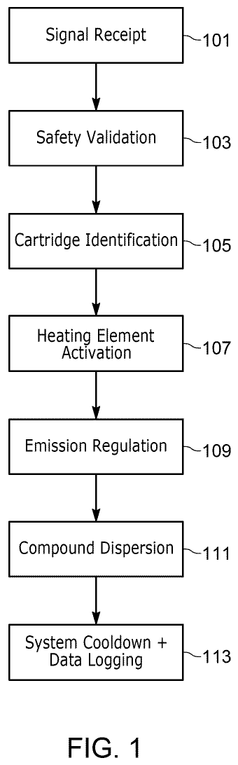

The following reference numerals are indicated in FIG. 1:

FIG. 1.101—The system receives an input signal that may originate from an external multimedia platform, a mobile application, a biometric sensor, or an internal scheduling module embedded in firmware. This signal initiates the emission sequence and may include parameters such as scent type, emission duration, or synchronization timing with other sensory outputs.

FIG. 1.103—Upon receipt of the signal, the control unit executes a validation routine that verifies operational readiness. This includes checking safety conditions such as housing temperature, cartridge status, and environmental constraints. If any failure or anomaly is detected, the emission sequence is aborted and a fault state may be logged.

FIG. 1.105—The control unit identifies the selected scent cartridge from among one or more available units. Cartridge identity may be verified through a tag (e.g., RFID or QR code), and availability is determined through physical or electrical presence detection. In systems supporting multiple aromas, the system maps the request to the appropriate cartridge channel.

FIG. 1.107—The system activates the heating element associated with the selected cartridge. Power is supplied in a controlled manner to achieve the thermal threshold required for volatilization of the aromatic compound. Heating profiles may be optimized to avoid degradation of scent integrity and to minimize power consumption.

FIG. 1.109—The control logic dynamically adjusts emission parameters, including the duration and intensity of heating, based on the input signal, environmental conditions, and pre-programmed profiles. Pulse-width modulation, duty cycling, or real-time feedback from onboard sensors may be used to fine-tune the output.

FIG. 1.111—Once volatilized, the aromatic compound is dispersed into the environment via passive diffusion or active airflow mechanisms. In embodiments with airflow control systems, micro-fans or directional vents guide the output toward the intended sensory zone or user.

FIG. 1.113—After the emission cycle concludes, the system deactivates the heating element and initiates a cooldown phase. Operational data including emission duration, cartridge usage, and sensor readings are logged for future diagnostics, performance optimization, or usage analytics.

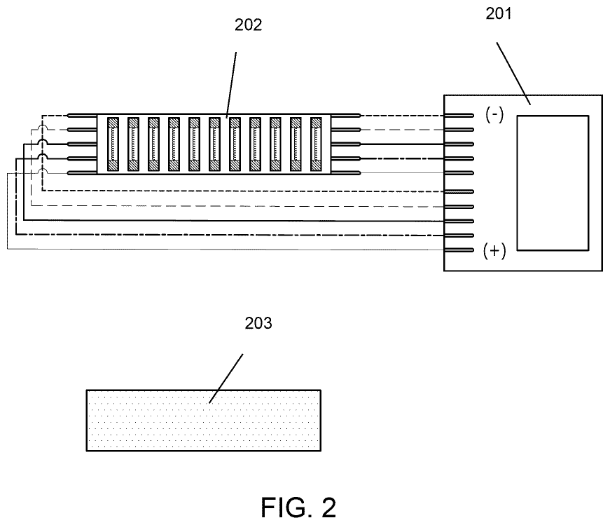

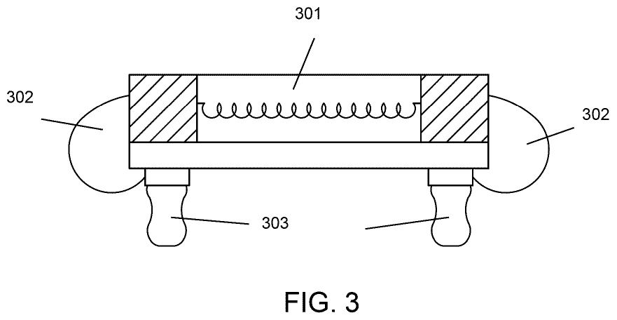

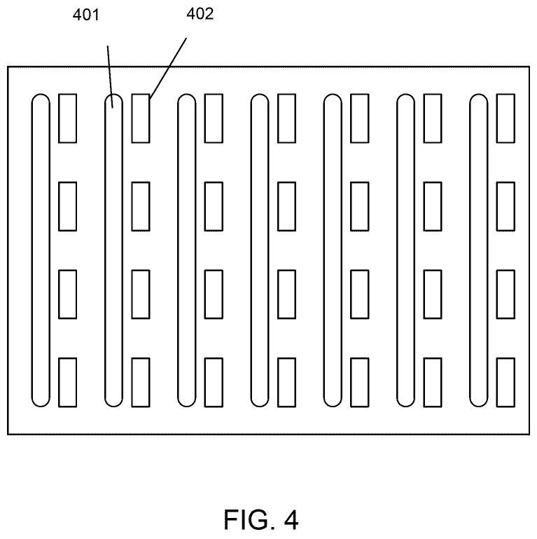

The following reference numerals are indicated in FIGS. 2-4:
201—power source
202—Diri
203—bandage covering
301—Diri
302—Wire connect
303—Post
401—flute
402—diri

What is claimed is:

1. An electrically actuated scent dispersion apparatus comprising:
a. a housing comprising a scent reservoir configured to hold an aromatic compound in at least one of a solid, gel, liquid, or encapsulated form;
b. at least one heating element operatively coupled to the scent reservoir and configured to volatilize the aromatic compound upon actuation;
c. a control unit electrically coupled to the heating element and configured to receive input signals from an external device or internal programming;
d, wherein the control unit regulates actuation of the heating element in accordance with a received or stored emission instruction to generate a controlled olfactory output;
and wherein the housing is dimensioned and structured for portable deployment or integration with an external platform selected from the group consisting of multimedia systems, wearable devices, assistive technologies, virtual reality environments, and medical instrumentation.

2. The apparatus of claim 1, wherein the control unit includes a microcontroller programmed to interpret digital signals corresponding to emission parameters including one or more of: emission duration, scent selection, thermal intensity, or repetition interval.

3. The apparatus of claim 1, wherein the heating element comprises a resistive heater selected from the group consisting of nichrome wire, printed conductive ink, metal oxide films, or etched foil heaters.

4. The apparatus of claim 1, further comprising a communication module selected from the group consisting of: Bluetooth, Wi-Fi, infrared (IR), near-field communication (NFC), universal serial bus (USB), or proprietary wireless protocols.

5. The apparatus of claim 1, wherein the scent reservoir is removably coupled to the housing and includes an identifier selected from the group consisting of RFID, QR code, EEPROM chip, or barcode for automatic recognition.

6. The apparatus of claim 1, wherein the housing further comprises one or more airflow control elements configured to direct volatilized aromatic compounds toward a target zone, said airflow control elements including at least one of a micro-fan, baffle, or vent aperture.

7. The apparatus of claim 1, further comprising at least one sensor selected from the group consisting of: proximity sensor, temperature sensor, motion detector, humidity sensor, or biometric sensor, wherein output from the sensor is used to modulate operation of the heating element.

8. The apparatus of claim 1, wherein the control unit includes onboard memory configured to store a scent emission profile and to execute an emission sequence in accordance with said profile without continuous external input.

9. The apparatus of claim 1, wherein the control unit is operatively connected to a host computing platform through an application programming interface (API), and wherein the host computing platform is configured to synchronize scent emission with a time-coded multimedia event.

10. The apparatus of claim 1, wherein the control unit is programmed to prevent activation of the heating element when a safety condition is detected, said safety condition comprising at least one of: excessive housing temperature, unrecognized cartridge, power instability, or restricted environment status.

11. The apparatus of claim 1, further comprising a plurality of scent reservoirs, each reservoir operatively coupled to a respective heating element, and wherein the control unit is configured to selectively activate one or more of said heating elements to emit a specific scent or combination thereof.

12. The apparatus of claim 11, wherein the plurality of scent reservoirs are arranged in a movable configuration selected from the group consisting of a rotating carousel, sliding tray, or electronically actuated selector mechanism.

13. The apparatus of claim 1, wherein the aromatic compound comprises a fragrance profile selected for therapeutic application, and wherein the control unit is configured to emit said fragrance profile in conjunction with a cognitive stimulation protocol.

14. The apparatus of claim 1, wherein the device is integrated with a mobile application operable on a smartphone, tablet, or wearable computing device, the mobile application providing user-configurable control over at least one of: scent intensity, emission timing, scent sequence, or content synchronization.

15. The apparatus of claim 1, wherein the housing includes an attachment mechanism selected from the group consisting of: adhesive backing, magnetic mount, clip, strap, or bracket configured for affixation to external platforms.
